(19) United States
(12) Patent Application Publication
(10) Pub. No.: US 2023/0136075 A1
(43) Pub. Date: May 4, 2023

(54) TREATMENT FOR SEPSIS-INDUCED ORGAN DYSFUNCTION

(71) Applicants: Rinaldo Bellomo, Ivanhoe East (AU); The Florey Institute of Neuroscience and Mental Health, Parkville (AU); Austin Health, Heidelberg (AU)

(72) Inventors: Rinaldo Bellomo, Ivanhoe East (AU); Yugeesh Ryan Lankadeva, Reservoir (AU); Clive Newton May, North Fitzroy (AU)

(21) Appl. No.: 17/995,700

(22) PCT Filed: Apr. 8, 2021
(86) PCT No.: PCT/AU2021/050321

§ 371 Date: Oct. 6, 2022

(30) Foreign Application Priority Data
Apr. 8, 2020 (AU) 2020901120

(51) Int. Cl. A61K 31/375; A61K 9/00; A61P 31/00; A61K 47/26

(52) U.S. Cl. CPC A61K 31/375; A61K 9/0019; A61P 31/00; A61K 47/26

(57) ABSTRACT

Provided herein are methods for treating or preventing a condition, and/or symptom or clinical manifestation, associated with systemic infection wherein a subject is intravenously administered ascorbic acid or a pharmaceutically acceptable salt, ester or isomer thereof in an amount of between about 700 mg/kg body weight and about 4,000 mg/kg body weight per day, optionally as a first bolus infusion followed by a subsequent continuous infusion. Also provided are methods comprising intravenously administering to the subject ascorbic acid or a pharmaceutically acceptable salt, ester or isomer thereof: (i) as a bolus dose of from about 350 mg/kg body weight to about 500 mg/kg body weight; and (ii) following (i), by continuous infusion over at least several hours at a dose of from about 60 mg/kg body weight/hr to about 500 mg/kg body weight/hr. Typically, the condition is sepsis or septic shock.

### FIELD OF THE ART

The present disclosure relates to methods for treating and preventing symptoms, manifestations and conditions associated with systemic infections, in particular bacterial, viral, fungal and polymicrobial infections. Particular embodiments relate to the treatment of sepsis and septic shock and of symptoms and manifestations thereof.

### BACKGROUND

Sepsis is a life-threatening pathophysiological syndrome characterised by an overwhelming host immune response to an underlying infection that can lead to multi-organ dysfunction and death. Toxins produced by an untreated or inadequately treated infection spill over into the bloodstream causing damage to multiple organs and tissues including the kidneys, brain, heart, lungs, spleen and liver.

Despite advances in modern hemodynamic, antibiotic, and ventilatory clinical support, sepsis represents a major clinical problem with no effective therapy and an alarmingly high death rate of 20-60%. It is the leading cause of death in intensive care units worldwide. Due to the increasing global incidence of sepsis (˜50 million cases/year), the annual mortality rate continues to rise (˜11 million deaths/year). Sepsis is more common and life threatening in individuals with a compromised immune system, and elderly individuals with pre-existing medical conditions (such as hypertension, diabetes, chronic kidney or liver disease, obesity, cancer, HIV) or injuries such as wounds or burns and/or in newborn babies with under-developed immune systems.

An initial hyperinflammatory process and subsequent immune paralysis contribute to mortality and morbidity in sepsis. The initial hyperinflammatory response is associated with uncontrolled cytokine production that can be deleterious to various tissues and can lead to organ injury and dysfunction. After this hyperinflammatory phase, an immune paralytic phase associated with enhanced apoptotic cell death occurs in multiple organs and tissues. Severe sepsis can result in septic shock, in which the systemic inflammatory response leads to the failure of vital organ function.

A common complication of sepsis is acute kidney injury which develops in up to 50% of septic patients who have an increased mortality rate. Currently there is no treatment for septic acute kidney injury, apart from renal replacement therapy which is both invasive and expensive.

Various viral, bacterial, fungal and parasitic infections can give rise to sepsis. Of particular concern is the continued emergence of viral diseases representing serious threats to human health, such as coronaviruses, a large family of single-stranded RNA viruses causing respiratory disease. In December 2019, a novel coronavirus emerged in China, designated severe acute respiratory syndrome coronavirus 2 (SARS-CoV-2). Extremely infectious, SARS-CoV-2 has infected more than 130 million people globally and resulted in over 2 8 million deaths so far. The clinical spectrum of the respiratory disease caused by SARS-CoV-2, COVID-19, varies from asymptomatic to severe clinical manifestations characterized respiratory failure (acute respiratory distress syndrome) necessitating ventilation support in an intensive care unit, acute lung inflammation, sepsis, septic shock and multiple organ dysfunction syndrome.

Standard of care treatment for sepsis consists of antibiotics, fluid resuscitation and vasopressors, with continuous renal replacement therapy being increasingly used in critically ill patients. These interventions are mostly aimed towards keeping the patient alive in the expectation that organ function should recover following resolution of the infection. However, patients who recover from severe sepsis frequently exhibit a degree of chronic organ dysfunction. Currently there are no treatments that reverse sepsis-induce organ dysfunction.

There is an urgent need for the development of clinically effective treatments for the clinical symptoms and manifestations of systemic infections such as sepsis and septic shock.

### SUMMARY OF THE DISCLOSURE

A first aspect of the present disclosure provides a method for treating or preventing a condition and/or symptom or clinical manifestation associated with systemic infection in a subject, comprising intravenously administering to the subject ascorbic acid or a pharmaceutically acceptable salt, ester or isomer thereof in an amount of between about 700 mg/kg body weight and about 4,000 mg/kg body weight per day.

In an exemplary embodiment, the ascorbic acid or pharmaceutically acceptable salt, ester or isomer thereof is administered in an amount of at least about 2,000 mg/kg body weight per day. In another exemplary embodiment, the ascorbic acid or pharmaceutically acceptable salt, ester or isomer thereof is administered in an amount of at least about 3,000 mg/kg body weight per day.

In an embodiment, the ascorbic acid or pharmaceutically acceptable salt, ester or isomer is administered in two or more doses per day, optionally over a period of several hours. In an exemplary embodiment, the two or more doses may be administered over a period of about six or seven hours.

In a particular embodiment, the daily amount of ascorbic acid or pharmaceutically acceptable salt, ester or isomer is administered in two doses, a first bolus infusion and a subsequent, second continuous infusion. The bolus infusion may be administered over a period of about 30 minutes. The continuous infusion may be administered for a period of about six to 24 hours, for example about six to twelve hours. In an exemplary embodiment, the bolus infusion comprises administration of from about 350 mg to about 500 mg ascorbic acid or pharmaceutically acceptable salt, ester or isomer per kg body weight, and the continuous infusion comprises administration of from about 60 mg to about 500 mg ascorbic acid or pharmaceutically acceptable salt, ester or isomer per kg body weight per hour, optionally for a period of about six or seven hours.

In an exemplary embodiment, the ascorbic acid is administered in the form of a pharmaceutically acceptable salt, ester or isomer, optionally sodium ascorbate.

In a particular embodiment, the condition is sepsis, septic shock or multiple organ dysfunction syndrome. The subject may have sepsis, septic shock or multiple organ dysfunction syndrome, or be at risk of developing sepsis, septic shock or multiple organ dysfunction syndrome.

In an embodiment, the systemic infection is a bacterial or viral infection. The bacterial infection may be caused by a gram-negative or gram-positive bacteria. The viral infection may be caused by a coronavirus. The coronavirus may be SARS-CoV-2. In an exemplary embodiment, the condition is COVID-19.

The symptom or clinical manifestation may be selected from reduced blood pressure, including requiring vasopressor support after fluid resuscitation, elevated heart rate, renal tissue hypoxia, renal tissue ischemia, sepsis-induced acute kidney injury, reduced urine output, cerebral tissue hypoxia, cerebral tissue ischemia, acute respiratory distress syndrome, hyperlactatemia, multiple organ dysfunction, or a combination of two or more of the foregoing.

A second aspect of the present disclosure provides a method for treating or preventing a condition and/or symptom or clinical manifestation associated with systemic infection in a subject, comprising intravenously administering to the subject ascorbic acid or a pharmaceutically acceptable salt, ester or isomer thereof:
(i) as a bolus dose of from about 350 mg/kg body weight to about 500 mg/kg body weight; and
(ii) following (i), by continuous infusion over at least several hours at a dose of from about 60 mg/kg body weight/hr to about 500 mg/kg body weight/hr.

The bolus infusion may be administered over a period of about 30 minutes. The continuous infusion may be administered for a period of about six to twelve hours, optionally six or seven hours.

In an exemplary embodiment, the pharmaceutically acceptable salt of ascorbic acid is sodium ascorbate.

In a particular embodiment, the condition is sepsis, septic shock or multiple organ dysfunction syndrome. The subject may have sepsis, septic shock or multiple organ dysfunction syndrome, or be at risk of developing sepsis, septic shock or multiple organ dysfunction syndrome.

In an embodiment, the systemic infection is a bacterial or viral infection. The bacterial infection may be caused by a gram-negative or gram-positive bacteria. The viral infection may be caused by coronavirus. The coronavirus may be SARS-CoV-2. In an exemplary embodiment, the condition is COVID-19.

The symptom or clinical manifestation may be selected from reduced blood pressure, including requiring vasopressor support after fluid resuscitation, elevated heart rate, renal tissue hypoxia, renal tissue ischemia, sepsis-induced acute kidney injury, reduced urine output, cerebral tissue hypoxia, cerebral tissue ischemia, acute respiratory distress syndrome, hyperlactatemia, multiple organ dysfunction, or a combination of two or more of the foregoing.

A third aspect of the present disclosure provides ascorbic acid or a pharmaceutically acceptable salt, ester or isomer thereof for use in a method of treating or preventing a condition and/or symptom or clinical manifestation associated with systemic infection in a subject, wherein the method comprises intravenous administration to the subject of between about 700 mg to about 4,000 mg ascorbic acid or pharmaceutically acceptable salt, ester or isomer per kg body weight per day.

A fourth aspect of the present disclosure provides ascorbic acid or a pharmaceutically acceptable salt, ester or isomer thereof for use in a method of treating or preventing a condition and/or symptom or clinical manifestation associated with systemic infection in a subject, wherein the method comprises intravenous administration to the subject of:
(i) a bolus dose of from about 350 mg to about 500 mg ascorbic acid or pharmaceutically acceptable salt, ester or isomer per kg body weight; and
(ii) following (i), continuous infusion over at least several hours at a dose of from about 60 mg to about 500 mg ascorbic acid or pharmaceutically acceptable salt, ester or isomer per kg body weight per hour.

### BRIEF DESCRIPTION OF THE DRAWINGS

Aspects and embodiments of the present disclosure are described herein, by way of non-limiting example only, with reference to the following drawings.

FIG. 1. Schematic representation of an exemplary intervention protocol in accordance with the present disclosure.

FIG. 2. Changes in systemic hemodynamics in response to sodium ascorbate (Na Asc) (closed squares, n=5) or vehicle (open circles, n=5) treatment during ovine sepsis and during recovery from Gram-negative infection. Mean arterial pressure (A), norepinephrine dose (B), cardiac output (C), heart rate (D), total peripheral conductance, and core temperature (F) during infusion of Escherichia coli from 0 to 31 hr of sepsis and then recovery over 48 hr following antibiotic therapy. All animals were initially resuscitated with fluid bolus therapy (Fluid BT, 30-mL/kg balanced crystalloid over 30 min) from 23.5 to 24 hr of sepsis. Animals were randomized to receive Na Asc (0.5 g/kg) or vehicle, crystalloid BT, from 24 to 24.5 hr of sepsis followed by an infusion of sodium ascorbate (0.5 g/kg/hr) or vehicle crystalloid from 24.5 to 31 hr of sepsis. Norepinephrine doses were titrated to maintain mean arterial pressure at baseline levels (75-80 mm Hg) from 25 to 31 hr of sepsis. All animals received IV antibiotics at 31 hr of sepsis (1-g ceftriaxone), with a repeated dose at 24 hr, and their recovery from infection was monitored over 48 hr. Time 0 is the mean of the 24th hr of baseline and times 23-31 hr of sepsis and 48 hr of recovery are means of 0.5-hr periods. Data are presented as treatment group-specific mean±standard error of mean. p values represent treatment-time interactions from a two-way repeated measures analysis of variance from 23 to 31 hr of Gram-negative sepsis. Following antibiotic therapy and cessation of Escherichia coli infusion, significant differences between the baseline (time 0) time point and the 16-, 24-, 40-, and 48-hr time points are indicated by *p <0.05 in the vehicle treatment group. p values represent the results of a Dunnett test using absolute values.

FIG. 3. Changes in renal hemodynamics, intrarenal tissue perfusion, and oxygenation in response to sodium ascorbate (Na Asc) (closed squares, n=5) or vehicle (open circles, n=5) treatment during ovine sepsis and during recovery from Gram-negative infection. Renal blood flow (A), renal vascular conductance (B), medullary perfusion (C), cortical perfusion (D), medullary oxygen tension (PO₂) (E), and cortical PO₂ (F) during infusion of Escherichia coli from 0 to 31 hr of sepsis and then recovery over 48 hr following antibiotic therapy. Fluid and drug infusions and statistical analyses are as detailed in FIG. 2. BT=bolus therapy.

FIG. 4. Changes in renal functional and plasma osmolar gap in response to sodium ascorbate (Na Asc) (closed squares, n=5) or vehicle (open circles, n=5) treatment during ovine sepsis and during recovery from Gram-negative infection. Urine output (A), plasma creatinine (B), creatinine clearance (C), fractional sodium excretion (D), plasma osmolar gap (E), and fractional potassium excretion (F) during infusion of Escherichia coli from 0 to 31 hr of sepsis and then recovery over 48 hr following antibiotic therapy. Significant differences between the baseline (time 0) time point and the 16-, 24-, 40-, and 48-hr time points are indicated by *p<0.05 in the vehicle-treatment group and #p<0.05 in the Na Asc—treatment group. Fluid and drug infusions and statistical analyses are as detailed in FIG. 2. BT=bolus therapy.

FIG. 5. Changes in arterial blood biochemistry in response to sodium ascorbate (Na Asc) (closed squares, n=5) or vehicle (open circles, n=5) treatment during ovine sepsis and during recovery from Gram-negative infection. Arterial blood lactate (A), arterial blood pH (B), oxygen tension (PO₂) (C), arterial blood sodium (D), partial pressure of carbon dioxide (PCO₂) (E), and arterial blood potassium (F) during infusion of Escherichia coli from 0 to 31 hr of sepsis and then recovery over 48 hr following antibiotic therapy. Significant differences between the baseline (time 0) time point and the 16-, 24-, 40-, and 48-hr time points are indicated by #p <0.05 in the Na Asc—treatment group. Fluid and drug infusions and statistical analyses are as detailed in FIG. 2. BT=bolus therapy.

FIG. 6. Changes in systemic hemodynamics and renal function in response to mega-dose sodium ascorbate (Na Asc) treatment in one septic human patient with a severe case of COVID-19 disease 2019 (n=1). Norepinephrine dose (A), arterial blood oxygen tension (PO₂) (filled squares) and inspired oxygen fraction (open circles) (B), mean arterial pressure (C), serum creatinine (D), heart rate (E), and urine output (F) are presented at pre-treatment (time 0), after a 30-min infusion of Na Asc bolus therapy (BT, 30 g), and then at hourly intervals during an infusion of Na Asc for 6.5 hr (4.6 g/hr).

FIG. 7. Noradrenaline dose requirements to achieve target blood pressure of 75-80 mm Hg (A) and mean arterial pressure (B) in septic sheep administered different doses of sodium ascorbate (1 g/kg—small squares; 2 g/kg—large squares; 3 g/kg—large circles) and in vehicle control sheep (small circles). BT=bolus therapy.

FIG. 8. Changes in arterial blood biochemistry (arterial blood PO2 (A), blood lactate (B) and body temperature (C) in septic sheep administered different doses of sodium ascorbate (1 g/kg—small squares; 2 g/kg—large squares; 3 g/kg—large circles) and in vehicle control sheep (small circles). BT=bolus therapy.

FIG. 9. Changes in renal function (medulla tissue PO₂ (A) and urine output (B)) in septic sheep administered different doses of sodium ascorbate (1 g/kg—small squares; 2 g/kg—large squares; 3 g/kg—large circles) and in vehicle control sheep (small circles). BT=bolus therapy.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

The articles “a” and “an” are used herein to refer to one or to more than one (i.e. , to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

As used herein the terms “treating”, “treatment”, “preventing”, “prevention” and grammatical equivalents refer to any and all uses which remedy, prevent, retard or delay the establishment of a condition, symptom or clinical manifestation associated with a system infection or sepsis, or otherwise prevent, hinder, retard, or reverse the progression of such a condition, symptom or clinical manifestation. Thus, the terms “treating” and “preventing” and the like are to be considered in their broadest context. For example, treatment does not necessarily imply that a patient is treated until total recovery. For example, where a condition displays or is characterized by multiple symptoms or manifestations, the treatment or prevention need not necessarily remedy, prevent, hinder, retard, or reverse all of said symptoms or manifestations, but may prevent, hinder, retard, or reverse one or more of said symptoms or manifestations.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal Even more preferably, the mammal is a human

The terms “norepinephrine” and “noradrenaline” are used interchangeably in the present specification.

The present inventors have developed and extensively studied a clinically relevant model of ovine hypotensive, hyperdynamic sepsis with acute kidney injury that has a similar phenotype to human sepsis. The inventors have demonstrated that there is a large decrease in renal medullary tissue perfusion and oxygenation very early in ovine sepsis (Calzavacca et al., 2015). The inventors have validated that reductions in renal medullary oxygenation are closely mirrored by reductions in bladder urinary oxygenation in ovine sepsis (Lankadeva et al., 2016; Lankadeva et al., 2018a) and human sepsis patients (Osawa et al., 2019). This supports the notion that renal medullary hypoxia occurs in a similar manner in both human and ovine sepsis-induced acute kidney injury, indicating similar mechanistic changes and supporting the validity of this animal model.

As described and exemplified herein the present inventors have utilised the above-mentioned ovine model of sepsis to demonstrate the surprising effectiveness of mega dose intravenous vitamin C (sodium ascorbate, 3.75 g/kg) administration to treat the symptoms and manifestations of sepsis, including improvements in attaining target blood pressure with significantly reduced vasopressor (norepinephrine) requirements, a decrease in heart rate, a reversal of tissue hypoxia in the renal medulla, a reversal of acute kidney injury (as indicated by significant increases in urine flow and reductions in plasma creatinine), and a reversal of ischemia and hypoxia in the brain. These results have been validated, as also exemplified herein, in a human patient with COVID-19, wherein mega dose intravenous vitamin C administration normalised blood pressure, reduced vasopressor requirements and improved renal function in less than six hours.

Prior to the present invention, the use of high doses of vitamin C in the treatment of sepsis has remained controversial, with efficacy unresolved. A prior study using a combination therapy of vitamin C (1.5 g 4 times/day; i.e. 0.1 g/kg/day) with hydrocortisone and thiamine, reduced organ failure and mortality from 40.4% to 8.5% (Marik et al., 2017). The authors of that study concluded that a daily dose of 6 g vitamin C combined with hydrocortisone over a four day period is optimal, and warned against the detrimental effects of intravenous mega-dose vitamin C, including worsening renal function. A subsequent multicentre randomised clinical trial found that a maximum dose of vitamin C of 6 g/day for up to 10 days with thiamine±hydrocortisone, had no significant benefits above placebo treatment (Fuji et al., 2020). As described herein, the present inventors have surprisingly found that a bolus intravenous infusion, followed by a continuous intravenous infusion of mega-dose intravenous vitamin C (60 to 150 g) is particularly effective in treating sepsis, including reversing multi-organ dysfunction, with no observable adverse side effects.

In one aspect, the present disclosure provides a method for treating or preventing a condition and/or symptom or clinical manifestation associated with systemic infection in a subject, comprising intravenously administering to the subject ascorbic acid or a pharmaceutically acceptable salt, ester or isomer thereof in an amount of between about 700 mg/kg body weight and about 4,000 mg/kg body weight per day.

In accordance with the present disclosure, the systemic infection may be any bacterial, viral, fungal, parasitic or polymicrobial infection. In exemplary embodiments described herein the infection may be a bacterial infection or a viral infection. Bacteria causing systemic infection and sepsis may be Gram-negative or Gram-positive, and may include, by way of example only, Escherichia coli, Klebsiella pneumonia, Pseudomonas aeruginosa, Bacteroides fragilis, Enterobacter spp., Proteus spp., Streptococcus pneumonia, Streptococcus pyogenes, Staphylococcus aureus and Enterococcus spp. Most viruses capable of infecting humans can give rise to system infection and sepsis, including for example enteroviruses, herpes simplex virus, influenza viruses, dengue viruses and coronaviruses. In an exemplary embodiment described herein, the systemic infection is caused by the SARS-CoV-2 coronavirus, the causative agent of COVID-19.

Particular embodiments of the present disclosure provide methods for treating or preventing sepsis, septic shock or multiple organ dysfunction in a subject.

As used herein, “sepsis” refers to a body's response to a systemic infection, characterized at least in part by a widespread inflammatory response. “Septic shock” refers to sepsis which has advanced to the point of a significant and persistent reduction in blood pressure and organ dysfunction, that is typically not responsive to intravenous fluid administration.

In accordance with the present disclosure, the subject may be known to have sepsis, be suspected of having sepsis or be determined to be likely to develop, or at risk of developing, sepsis.

As will be well known to those skilled in the art, sepsis may be diagnosed in a variety of ways. For example, sepsis may be diagnosed by the presence of two or more of the following four systemic inflammatory response syndrome (SIRS) criteria: tachycardia (heart rate>90 bpm); hyperventilation (respiratory frequency>20 breaths/min); fever (greater than 38.3° C.) or hypothermia (less than 36° C.); and leukocytosis leukopenia, or bandemia (white blood cells greater than 1,200/mm³, less than 4,000/mm³ or bandemia≥10%). Alternatively, sepsis may be diagnosed using the sequential organ failure assessment (SOFA), or quick SOFA, score (see, e.g. Singer et al., 2016). Alternatively, or in addition, the presence of sepsis may be determined using one or more blood tests, for example for CBC complement, CFC and/or serum lactate levels. The skilled addressee will appreciate that the scope of the present disclosure is not limited by reference to any specific means or method of diagnosing sepsis in a subject.

Embodiments of the present disclosure provide methods for treating one or more symptoms or clinical manifestations of systemic infections, sepsis, septic shock and associated conditions. Such methods thereby provide means to remedy, improve or reverse one or more symptoms or clinical manifestations. By way of example, suitable symptoms and clinical manifestations to which the present methods may be applicable include reduced blood pressure (including requiring vasopressor administration to maintain target blood pressure), elevated heart rate, renal tissue hypoxia, renal tissue ischemia, sepsis-induced acute kidney injury, reduced urine output, cerebral tissue hypoxia, cerebral tissue ischemia, acute respiratory distress syndrome, hyperlactatemia or multiple organ dysfunction, or a combination of two or more of the foregoing.

The methods of the present disclosure provide for the administration of ascorbic acid or a pharmaceutically acceptable salt, ester or isomer thereof. The ascorbic acid may be in any suitable form, for example an isomeric form such as, but not limited to, L-ascorbic acid, D-ascorbic acid, L-isoascorbic acid or D-isoascorbic acid. Pharmaceutically acceptable salts of ascorbic acid that may be employed include, but are not limited to, sodium ascorbate, calcium ascorbate, magnesium ascorbate, and potassium ascorbate, sodium and potassium ascorbate or combinations thereof. Pharmaceutically acceptable esters of ascorbic acid that may be employed include, but are not limited to, ascorbyl phosphate, ascorbyl palmitate and ascorbyl stearate, or a combination thereof. For example, ascorbyl phosphate esters can include, but are not limited to mono, di, and tri sodium phosphates, magnesium phosphates, and calcium salt phosphates. Compositions and formulations for use in accordance with the present disclosure may comprise a single form, or multiple forms, of ascorbic acid or pharmaceutically acceptable salt, ester or isomer thereof. Similarly, the ascorbic acid or pharmaceutically acceptable salt, ester or isomer thereof may be provided, obtained or derived from a single source or multiple sources, and may be provided, obtained or derived from one or more natural and/or synthetic sources.

In a particular exemplary embodiment of the present disclosure sodium ascorbate is administered. However, the skilled addressee will appreciate that the scope of the present disclosure is not to be limited by reference to any specific form or ascorbic acid or salt, ester or isomer thereof.

The methods of the present disclosure provide for the intravenous administration of the ascorbic acid or pharmaceutically acceptable salt, ester or isomer thereof, in an amount of between about 700 mg/kg body weight and about 4,000 mg/kg body weight per day. Typically the intravenous administration is performed via an infusion pump, as will be well known to those skilled in the art, although the scope of the present disclosure is not limited by reference to any specific means of intravenous administration. The suitable means can be determined by the skilled person depending on the amount of ascorbic acid, or salt, ester or isomer thereof, to be administered, the length of time the administration is to continue and the desired rate of administration.

Pharmaceutical forms of ascorbic acid and salts, esters and isomers thereof, suitable for intravenous injection include sterile aqueous solutions or dispersions and sterile powders for the extemporaneous preparation of sterile injectable solutions or dispersions. The formulation should be stable under the conditions of manufacture and storage and be preserved against the contaminating action of microorganisms such as bacteria, viruses and fungi. The formulation, or the package or container (e.g. sterile bag or pouch) in which the formulation is stored or maintained prior to use should ideally also protect the ascorbic acid or salt, ester or isomer thereof from oxidation, for example by minimising exposure to light, UV light, high temperature, metals and/or oxygen.

Compositions comprising the ascorbic acid or salt, ester or isomer thereof can be formulated in any dosage forms that are suitable for intravenous administration, including solutions, suspensions, and solid forms (e.g. powders) suitable for preparing solutions or suspensions in liquid prior to injection. Such dosage forms can be prepared according to conventional methods known to those skilled in the art of pharmaceutical science (see, e.g., Remington: The Science and Practice of Pharmacy, supra). Compositions can include one or more pharmaceutically acceptable carrier(s) and excipient(s), including, but not limited to, aqueous vehicles, water-miscible vehicles, non-aqueous vehicles, antimicrobial agents or preservatives, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, and pH adjusting agents. Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants include, but are not limited to, bisulfite and sodium metabisulfite. Suitable local anesthetics include, but are not limited to, procaine hydrochloride. Suitable suspending and dispersing agents include, but are not limited to, sodium carboxymethylcelluose, hydroxypropyl methylcellulose, and polyvinylpyrrolidone. Suitable emulsifying agents include, but are not limited to, polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin.

Compositions comprising the ascorbic acid or salt, ester or isomer thereof for use in accordance with the present disclosure may be provided in a form to be reconstituted prior to intravenous delivery, may be provided in a form requiring dilution or mixing prior to use, or may be provided in a ready-to-use form (i.e. in a form not requiring an intervening step of reconstitution, dilution or mixing prior to administration). Thus, contemplated and envisaged herein is the provision of high dose vitamin C formulations in sterile infusion packs, such as bags or pouches. Infusion packs of any suitable volume may be provided, such as from about 100 mL to about 2 L, optionally from about 200 mL to about 1 L. The skilled person will appreciate that the form of composition or formulation provided, and the package or container in which the composition or formulation is stored or placed for use is not limiting on the scope of the present disclosure. Any suitable composition or formulation, package or container known to those skilled in art is contemplated herein and should be regarded as falling within the scope of the present disclosure.

The precise amount of ascorbic acid, or salt, ester or isomer thereof, to be administered can be determined by the skilled person based on a variety of factors including the age of the subject, the severity of the systemic infection and/or sepsis or associated condition suffered by the individual, the medical history of the subject including any co-morbidities, the body weight of the subject, age of the subject, and the form in which the ascorbic acid is administered. The amount should be a “therapeutically effective amount”, being an amount sufficient to provide the desired therapeutic effect. Thus, it is not possible to specify an exact “effective amount”. However, for any given case, an appropriate “effective amount” may be determined by one of ordinary skill in the art using only routine experimentation.

The amount of ascorbic acid or salt, ester or isomer thereof is between about 700 mg/kg body weight and about 4000 mg/kg body weight per day. For example, the amount may be about 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg or 4000 mg per kg body weight per day.

The daily amount of from about 700 to about 4000 mg ascorbic acid or salt, ester or isomer thereof per kg body weight may be administered in a single dose, but more typically in two or more doses. Where multiple dosed are administered per day, each dose may comprise the same or different amounts of the ascorbic acid or salt, ester or isomer thereof. Moreover, each dose may comprise the same or different forms of the ascorbic acid or salt, ester or isomer thereof. The doses may be administered over one or more hours, for example about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, or 48 hours. Methods of the present disclosure may comprise administration in a single day (i.e. over one or more hours on a single day). Alternatively administration of the stated daily amount may be repeated (over one or more hours as described above) on one or more subsequent days, as determined by the skilled person on a case by case basis. Where administration is continued for multiple days, these days may or may not be consecutive, again as determined by the skilled person on a case by case basis. The skilled person will also appreciate that where administration is continued for multiple hours or days, the amount or concentration of ascorbic acid or salt, ester or isomer thereof administer may be reduced over the course of the treatment.

In a particular exemplary embodiment described herein, the daily amount of ascorbic acid or pharmaceutically acceptable salt, ester or isomer is administered in at least two doses, comprising one or more bolus infusions followed by one or more subsequent, continuous infusions. The bolus infusion(s) may typically be administered over a period of between about 10 minutes and 60 minutes, for example over a period of about 10 minutes, 20 minutes, 30 minutes, 40 minutes, 50 minutes or 60 minutes. The continuous infusion(s) may be administered for a period of one or more hours, for example about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, or 48 hours. . In an exemplary embodiment, administration in accordance with the present disclosure comprises a first bolus infusion and a subsequent, second continuous infusion, wherein the bolus infusion is administered over a period of about 30 minutes and the continuous infusion is administered for a period of about six or seven hours.

In an exemplary embodiment, the bolus infusion comprises administration of from about 350 mg to about 500 mg ascorbic acid or pharmaceutically acceptable salt, ester or isomer per kg body weight, and the continuous infusion comprises administration of from about 60 mg to about 500 mg ascorbic acid or pharmaceutically acceptable salt, ester or isomer per kg body weight per hour, optionally for a period of about six or seven hours. The bolus infusion may comprise, for example, about 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg ascorbic acid or pharmaceutically acceptable salt, ester or isomer per kg body weight of the subject. The continuous infusion may comprise, for example, about 60 mg, 80 mg, 100 mg, 125 mg, 150 mg, 175 mg, 200 mg, 225 mg, 250 mg, 275 mg, 300 mg, 325 mg, 350 mg,375 mg, 400 mg, 425 mg, 450 mg, 475 mg or 500 mg ascorbic acid or pharmaceutically acceptable salt, ester or isomer per kg body weight per hour, optionally for a period of about six or seven hours.

Accordingly, in one aspect the present disclosure provides a method for treating or preventing a condition and/or symptom or clinical manifestation associated with systemic infection in a subject, comprising intravenously administering to the subject ascorbic acid or a pharmaceutically acceptable salt, ester or isomer thereof:
(i) as a bolus dose of from about 350 mg/kg body weight to about 500 mg/kg body weight; and
(ii) following (i), by continuous infusion over at least several hours at a dose of from about 60 mg/kg body weight/hr to about 500 mg/kg body weight/hr.

In order to increase the effectiveness of the methods of the present disclosure, it may be desirable to combine the administration of ascorbic acid or pharmaceutically acceptable salt, ester or isomer thereof with one or more additional agents effective in the management, treatment or prevention of sepsis, septic shock and related conditions, or for managing or improving one or more symptoms or clinical manifestations thereof. Such additional agents may be administered in the same formulation as the ascorbic acid or salt, ester or isomer thereof, or in a different formulation(s), administered via the same or different routes. Such administration may be simultaneous with, or sequential to, the ascorbic acid, salt, ester or isomer administration. In this context, “sequential” administration is meant a time difference of from seconds, minutes, hours or days between the administration of the agents.

By way of example, agents that may be administered in conjunction with the ascorbic acid or salt, ester or isomer thereof, in accordance with the present disclosure include for example vasopressors such as norepinephrine, a glucocorticoid, thiamine, steroidal and non-steroidal anti-inflammatory agents, and agents to treat the cause of the underlying systemic infection, including antimicrobial agents, antibiotics, antiviral agents, antifungal agents and anti-parasitic agents.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

The following examples are illustrative of the disclosure and should not be construed as limiting in any way the general nature of the disclosure of the description throughout this specification.

### Example 1—High Dose Intravenous Vitamin C Therapy in an Oovine Model of Sepsis

As described hereinbefore, the inventors have developed a clinically relevant model of ovine hypotensive, hyperdynamic sepsis with acute kidney injury that has a similar phenotype to human sepsis (see e.g. Calzavacca et al., 2015). Using this model, the inventors have investigated the effect of mega dose intravenous vitamin C therapy on various parameters indicative of the progression of sepsis in sheep (Merino ewes, 35-45 kg; 1.5-2.0 years old). Sheep were divided into two groups, a vitamin C (sodium ascorbate) therapy group and a vehicle group as a control. Experimental protocols were approved by the Animal Ethics Committee of the Florey Institute of Neuroscience and Mental Health under guidelines of the National Health and Medical Research Council of Australia.

Preparatory surgical procedures were carried out as described in Lankadeva et al. (2018b). Briefly, two preparatory surgical procedures were performed under general anaesthesia. First, a carotid artery was exteriorised into a skin fold to form a carotid arterial loop to facilitate cannulation for measurement of arterial pressure and heart rate, and for blood sampling. During the same surgical procedure, a flow probe was placed around the pulmonary artery to measure cardiac output. Animals were allowed 3-4 weeks of recovery. Second, a flow probe was implanted around a renal artery to measure renal blood flow, a renal vein was cannulated for blood sampling and fibre optic probes were inserted into the renal cortex, renal medulla (within kidneys) and parietal cortex (brain) to measure tissue perfusion, oxygen tension and temperature.

During the second surgical procedure cannulae were implanted in the carotid artery for arterial pressure measurement and blood sampling and the jugular vein for infusion of fluids, vasopressors, Escherichia coli, sodium ascorbate and blood sampling, and a Foley catheter was inserted into the bladder to collect urine and monitor hourly urine output.

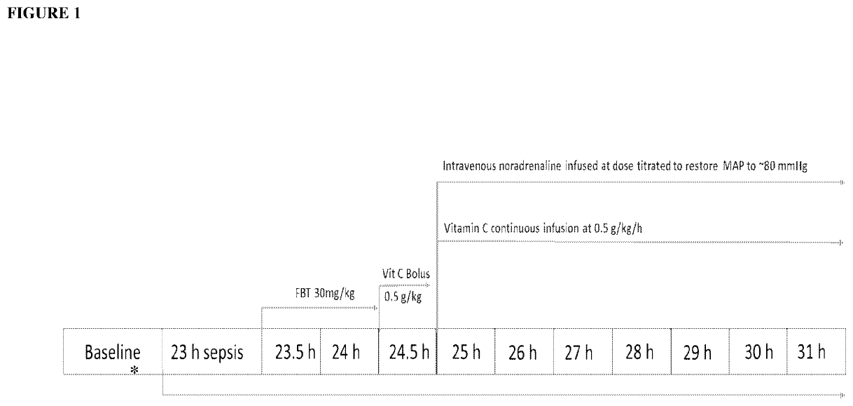

Following three days of recovery from the second surgical procedure, the experimental protocol (see FIG. 1) was initiated in conscious sheep, to avoid the confounding effects of general anaesthesia. Animals daily food and water intake and urine output was measured on a 24-hour basis during the course of the 5-day protocol.

First, a 24-hour baseline period was commenced, which included measurement of systemic haemodynamics, renal and brain perfusion and oxygenation, arterial, central venous and renal venous blood gases and lactate, and urine output. Arterial blood and urine samples were collected at set times during the experimental period.

After establishment of the 24-hour baseline period, gram-negative sepsis was induced by intravenous infusion of live E. coli (isolated from a patient with sepsis at Austin Health) as described in Lankadeva et al. (2018b), administered as a loading (bolus) infusion of 2.8×10⁹ colony-forming units (CFU) over 30-minutes followed by a continuous infusion of 1.26×10⁹ CFU/h for 30.5 hours. No prophylactic antibiotics, sedative or analgesic agents were administered during this protocol. The description in the following paragraphs is with reference to the protocol as shown schematically in FIG. 1.

At 23 hours of sepsis, when sheep had fulfilled the clinical criteria for sepsis (i.e. established hypotension, tachycardia, fever, lung injury, tachypnoea, hyperlactatemia and stage 1-2 acute kidney injury) animals were resuscitated with 30 mL/kg balanced crystalloid sodium lactate (Hartmann's solution infused at approximately 2500 ml/hr) for 30 mins, from 23-23.5 hours of sepsis.

A blocked randomization design was used to allocate sheep to treatment with sodium ascorbate or vehicle. From 24.0-24.5 hours of sepsis, animals in the sodium ascorbate group (n=5) received an intravenous loading infusion of sodium ascorbate (Biological Therapies, AU; 30 g in 100 ml water; Biological Therapies) at a dose of 0.5 g/kg body weight (20 g sodium ascorbate for a 40 kg sheep) and an infusion rate of approximately 400 ml/hr. From 24.5-31 hours of sepsis, animals received a maintenance (continuous) infusion of sodium ascorbate at a dosage rate of 0.5 g/kg body weight/hr (20 g/hour for a 40 kg sheep) and an infusion rate of approximately 200 ml/hr. In the vehicle group (n=5), fluid-matched Hartmann solution was administered from 24 to 31 hours of sepsis.

In animals of both groups, at 25 hours of sepsis, the primary clinical vasopressor, norepinephrine (Hospira, AU) was infused at a dose to return mean arterial pressure to baseline values (˜80 mm Hg). Norepinephrine was discontinued if an animal was able to maintain its own blood pressure at the target of 80 mmHg

At 31 hours of sepsis, the sodium ascorbate and E. coli infusions were terminated, and 1 g of Ceftriaxone (clinical non-nephrotoxic antibiotic for gram-negative infections) was administered intravenously. Systemic haemodynamics, and renal and brain perfusion and oxygenation were then continuously monitored over a 48-hour recovery period, with repeated intravenous doses of Ceftriaxone (1 g) at 24-hour intervals. All animals received a maintenance infusion of Hartmann solution (1 mL/kg/hr) during the 48 hours of recovery. At 48 hours of recovery, animals were humanely euthanised with an overdose of sodium thiopentone and the brain and kidneys were collected for histological assessment.

At baseline and at regular intervals post induction of sepsis, systemic hemodynamic responses and renal responses were measured according to the methods described in Lankadeva et al. (2018b).

Fibre-optic probes were implanted into the brains of the sheep for the measurement of tissue perfusion and oxygenation. An incision was made in the scalp 1.0 cm lateral to the sagittal suture, 5.0 cm in length, with the caudal end of the incision at the level of bregma. The periosteum was removed and a small craniotomy (2.0 mm diameter) was made 1.0 cm lateral to the sagittal suture and 1.0 cm rostral to bregma. A custom-built fibre-optic probe (CP-004-001. Oxford Optronix, Oxford, UK), with 20 mm of optical fibre extending from the outer sheath, was inserted into the brain to measure tissue perfusion and oxygenation. The probe was prepared with a flexible sheet glued to the outer sheath, as described previously for insertion of these probes into the kidney (Calzavacca et al., 2015). The probe, pointed rostrally at an angle of 60° and parallel to the sagittal suture, was inserted into the brain along a tract previously made by insertion of a 25 G needle through the dura into the brain. The flexible sheet on the probe was secured to the skull with cyanoacrylate adhesive and the scalp was sutured. At post-mortem, it was observed that the tip of the probe was 10-15 mm from the surface of the brain in the parietal cortex.

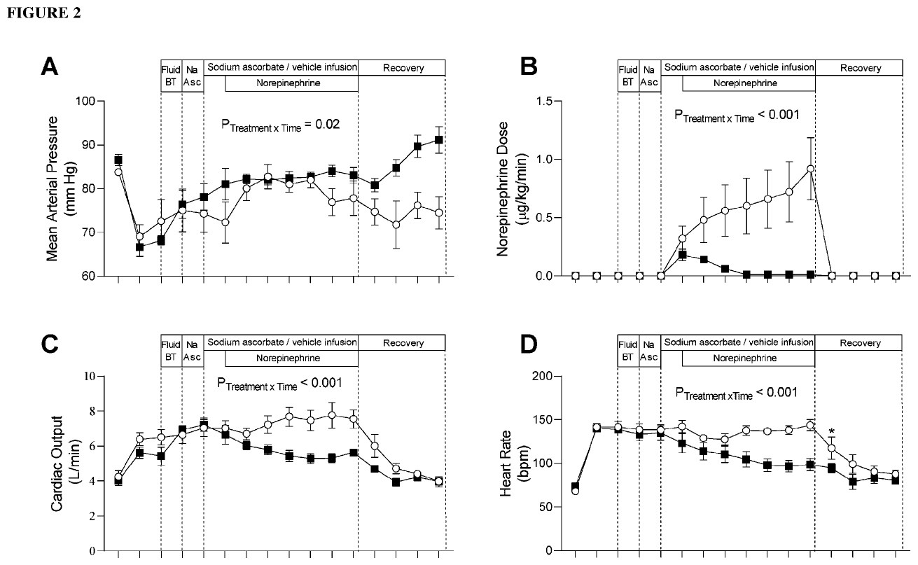

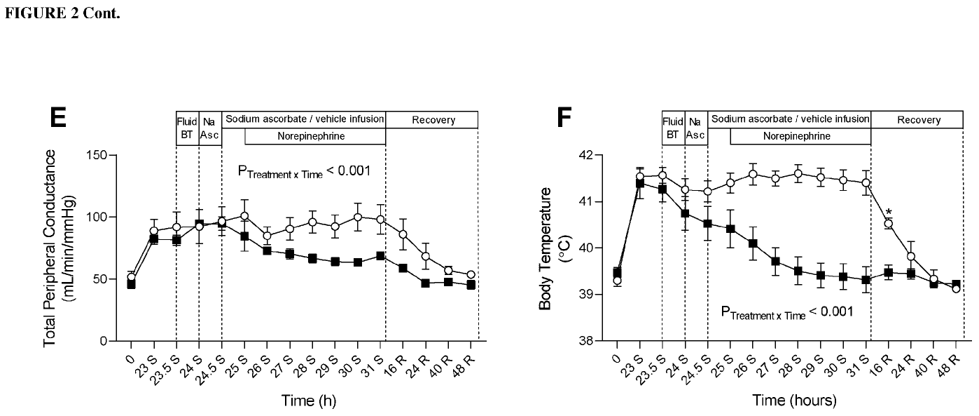

Septic sheep demonstrated malaise and lethargy, were drowsy and unresponsive to external stimuli, mostly lay down, and did not eat or drink. They all developed a persistent high fever (41.4±0.2° C.) and tachycardia (141±2 beats/min) (FIGS. 2D and 2F). Fluid bolus therapy had no effect on this clinical state. After infusion of sodium ascorbate for 3 hours, the clinical state of all sheep dramatically improved. They stood up, were alert and responsive to external stimuli, and began to drink water and eat. They looked well and similar to a normal, healthy animal This improvement in clinical condition remained during the sodium ascorbate infusion and the 2 recovery days. Furthermore, body temperature decreased to normal levels (˜39° C. in sheep) during sodium ascorbate therapy (FIG. 2F).

The septic clinical state was associated with a hypotensive, hyperdynamic circulatory state and stage 1 acute kidney injury. The deterioration in renal function occurred despite increased renal blood flow and increased renal oxygen delivery. Renal hyperemia was associated with increased renal cortical tissue perfusion and PO₂, but large reductions in renal medullary tissue perfusion and PO₂. Sheep also developed moderate arterial hypoxemia (Pao2˜80 mm Hg) and hyperlactatemia (˜2.0 mmol/L).

Fluid bolus therapy caused a small improvement in mean arterial pressure and further increased cardiac output (FIGS. 2A and C). During the subsequent infusion of sodium ascorbate, from 24 to 31 hours of sepsis, the dose of norepinephrine required to maintain mean arterial pressure progressively decreased, such that by 4 hours of sodium ascorbate infusion, norepinephrine was not required in four of five sheep (FIG. 2B). Despite the reduced norepinephrine dose, to zero in four of the five sheep, the level of peripheral vasoconstriction increased, accompanied by significant, progressive decreases in cardiac output and heart rate toward baseline values (FIG. 2C-E). In contrast, in the vehicle group, the dose of norepinephrine had to be continuously increased, and by 30 hours of sepsis, responsiveness to norepinephrine had declined to a such degree that it was not possible to maintain the target mean arterial pressure (FIG. 2A).

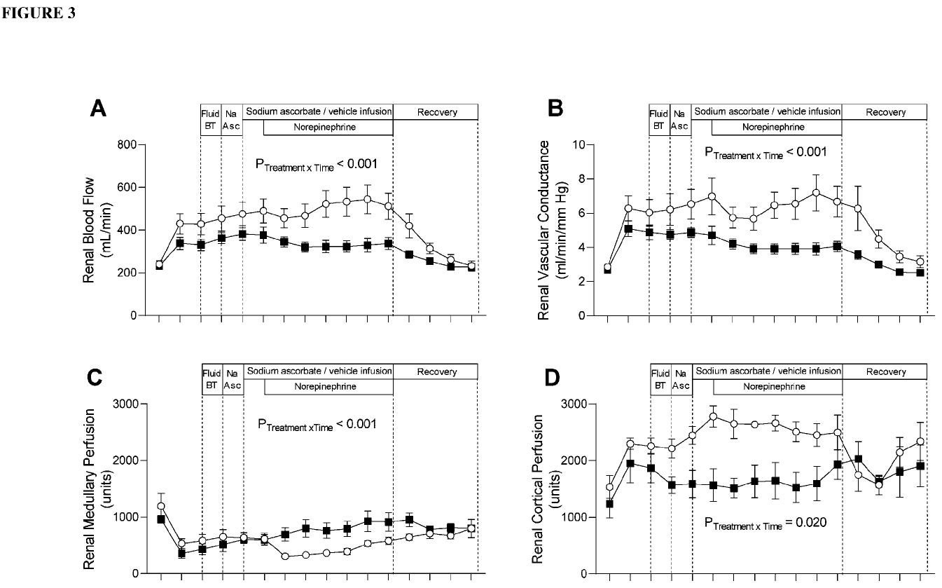

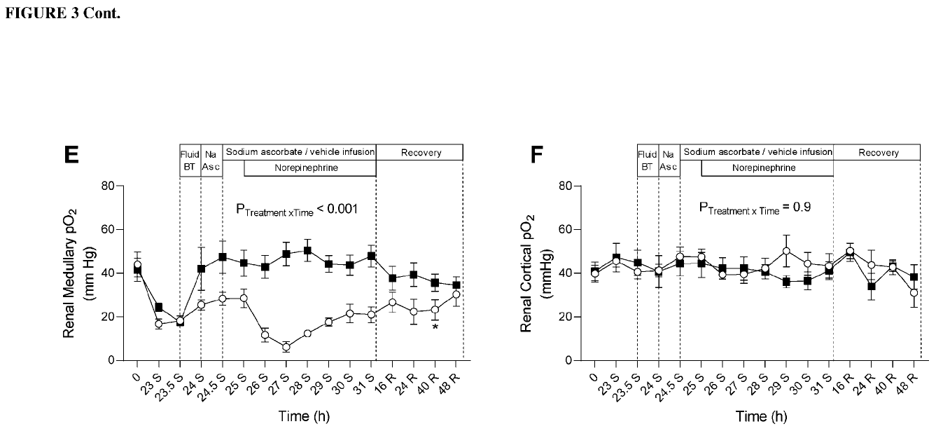

Sepsis caused increases in renal blood flow and renal vascular conductance that were maintained from 24 to 31 hours of sepsis in the vehicle-treated group, whereas both renal blood flow and renal vascular conductance decreased toward healthy levels in the sodium ascorbate-treated group (FIG. 3A and B). Fluid bolus therapy transiently improved both medullary perfusion and PO₂, but the levels decreased during infusion of vehicle and norepinephrine (FIGS. 3C and E). In contrast, with sodium ascorbate, renal medullary perfusion improved toward preseptic levels and medullary PO₂ increased further throughout the infusion (FIGS. 3C and E). These beneficial changes were maintained for the following 2 days. The elevations in renal cortical perfusion observed in the vehicle group after fluid and norepinephrine therapy were absent in sheep treated with sodium ascorbate (FIG. 3D). Neither fluid bolus therapy, sodium ascorbate, nor norepinephrine had significant effects on renal cortical tissue PO₂ (FIG. 3F).

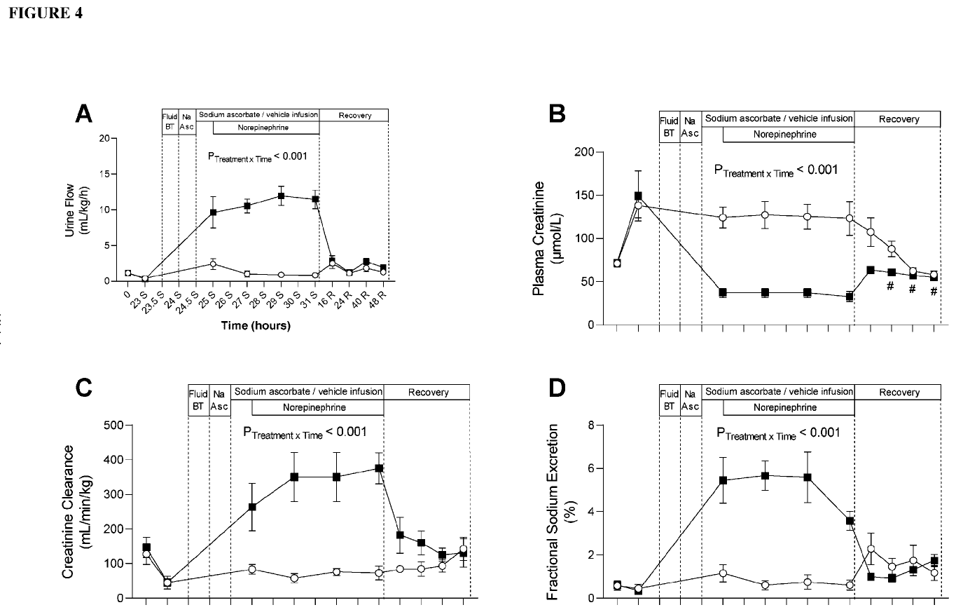

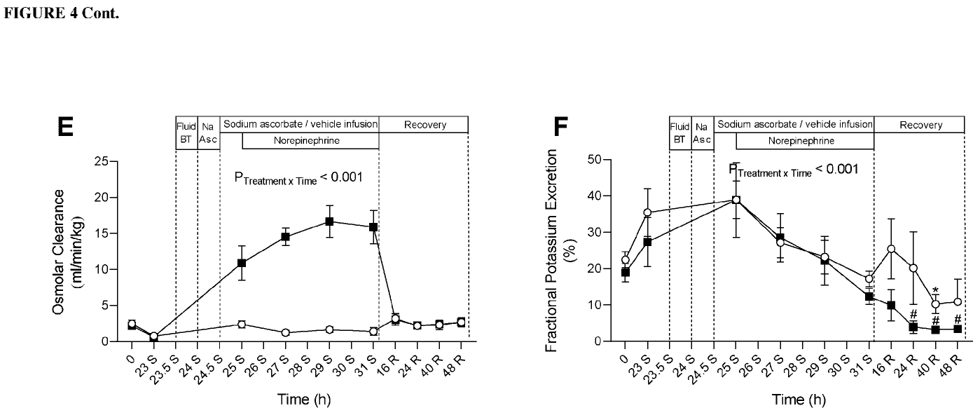

In septic sheep, infusion of sodium ascorbate dramatically increased urine flow, to greater than 10 mL/kg/hr, within 30 minutes. Urine flow remained at these high levels throughout the infusion (FIG. 4A). The following 2 days, urine flow remained at normal baseline levels. Treatment with sodium ascorbate corrected the increased plasma creatinine to below baseline levels (FIG. 4B) and significantly increased creatinine clearance and fractional excretion of sodium (FIGS. 4C and D), but did not alter fraction excretion of potassium (FIG. 4F). Consistent with the predicted high levels of vitamin C in plasma, the plasma osmolar clearance increased (FIG. 4E). In contrast, in the vehicle group, acute kidney injury persisted: plasma creatinine remained elevated, and creatinine clearance and urine flow remained at low levels (FIG. 4).

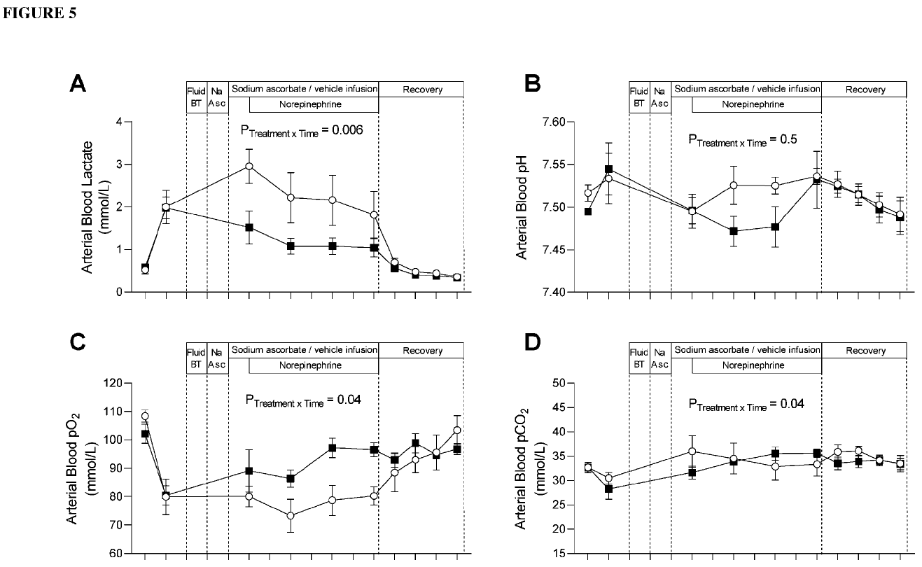

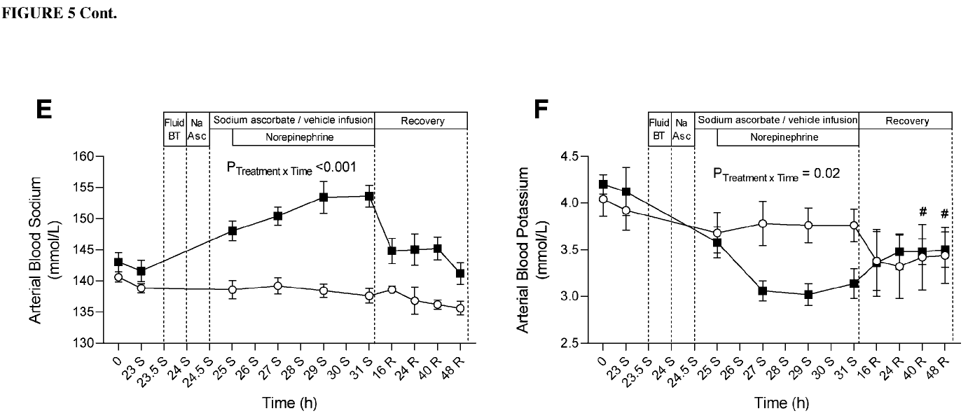

By 23 hours of sepsis, arterial lactate had tripled in both groups (0.6±0.1 to 2.0±0.2 mmol/L). In the vehicle- treated group, arterial lactate remained increased throughout the intervention period. In contrast, with sodium ascorbate, there was a significant, progressive reduction in lactate (FIG. 5A). Arterial PO₂ was significantly reduced at 23 hours of sepsis (102±2 to 80±3 mm Hg) and remained at this moderately hypoxic level during infusion with vehicle. In contrast, sodium ascorbate progressively improved arterial PO₂ (to 96.5±3.4 mm Hg) (FIG. 5C), but had no effects on pH or partial pressure of carbon dioxide (FIG. 5B and D). Arterial blood levels of sodium and potassium were unchanged by sepsis or infusion of vehicle. Infusion of sodium ascorbate, however, caused hypernatremia (153.6±1.7 mmol/L) and hypokalemia (3.14±0.16 mmol/L) (FIGS. 5E and F). Potassium chloride (16 mmol/hr) was infused if arterial potassium decreased below 2.5 mmol/L.

The increases in plasma bilirubin and plasma aspartate aminotransferase during sepsis were significantly reduced by sodium ascorbate. There was no acute tubular necrosis or interstitial fibrosis within the renal cortex, corticomedullary junction, or medulla in either treatment group.

To examine the effect of administering an equivalent amount of sodium to that in the sodium ascorbate infusion described above, using a similar protocol, two septic sheep were infused with a loading dose of NaHCO₃ (8.4 g/kg over 30 minutes) followed by a sodium bicarbonate infusion over 6.5 hours (at 8.4 g/kg/hr).

Infusion of hypertonic NaHCO₃, at a dose to equal the sodium load with sodium ascorbate, did not reproduce the effects of sodium ascorbate (data not shown): MAP decreased and increasing doses of norepinephrine were required, heart rate and RBF were not reduced, there was no improvement in arterial PO₂ or renal medullary perfusion or PO₂, and no reduction in plasma creatinine or increase in creatinine clearance was observed, although urine flow increased. There was a large increase in blood pH, with hypernatremia and hypokalemia. During the infusion of NaHCO3, there was intermittent shivering and large increases in body temperature.

### Example 2—High Dose Intravenous Vitamin C Therapy in a COVID-19 Patient

A 40-year old male was admitted to ICU with severe COVID-19 disease. The patient presented with acute respiratory distress syndrome (ARDS), vasodilatory shock and acute kidney injury, with a temperature of 40° C. The patient was intubated and placed on a ventilator. At noon, his urinary output was 10 ml/hr.

The patient (weighing approximately 80 kg) was administered 30 g of intravenous sodium ascorbate over 30 minutes beginning at 1 pm, followed by an infusion of 30 g sodium ascorbate over the next 6 hours.

Over the course of the sodium ascorbate administration, renal function improved with plasma creatinine levels reducing from 118 μmol/L to 84 μmol/L, and urinary output progressively increased:
Prior to sodium ascorbate treatment: 10 ml/hr
1^{st} hour post initiation of treatment: 175 ml/h
2^{nd} hour post initiation of treatment: 160 ml/h
3^{rd} hour post initiation of treatment: 400 ml/h
4^{th} hour post initiation of treatment: 180 ml/h
5^{th} hour post initiation of treatment: 125 ml/h
6^{th} hour post initiation of treatment: 145 ml/h

The patient did receive three intermittent boluses of furosemide, but it is well established that the improvements in urinary output with this drug in the presence of acute kidney injury is transient and does not produce the sustained improvements observed here in the presence of sodium ascorbate.

Over the course of the sodium ascorbate administration, norepinephrine infusion decreased from 8 μg/min to 0, reflecting a normalisation of arterial pressure with reduced vasopressor support, similar to that observed in the sheep model (Example 1).

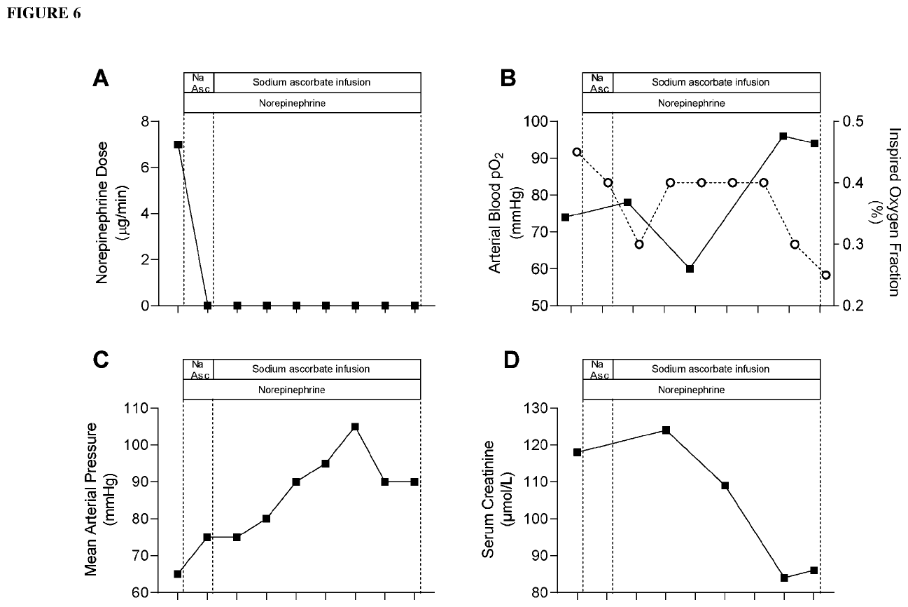

Arterial blood oxygen levels improved from 65 mm Hg (pre-treatment) to 90 mm Hg (after 6 hours of sodium ascorbate treatment) at the same level of supplemental oxygen (FiO₂:40%) without restarting vasopressors or fluid bolus therapy (FIG. 6A, 6C). Plasma creatinine decreased (118-84 μmol/L), whereas urine flow increased (10-400 mL/hr) during the treatment (FIG. 6D, 6F). Heart rate dropped (130-105 beats/min) (FIG. 6E) and lactate decreased (2.6-1.9 mmol/L). Arterial PO₂ increased, although FiO₂ dropped (0.45-0.30) with the same setting of positive end-expiratory pressure (14 cm H₂O) and without prone-positioning (FIG. 6B). The patient was extubated on intensive care day 15 (12 days after the treatment) and discharged from hospital without any complications at 22 days after treatment.

### Example 3—Vitamin C Dose Response in Ovine Sepsis

The inventors then sought to determine the minimum dose of intravenous vitamin C required to reverse the pathophysiological features of sepsis and acute kidney injury, using the ovine sepsis model described in Example 1. Surgical procedures and initiation of gram negative sepsis were as described in Example 1.

At 24 hours of sepsis, sheep were randomised into one of four groups (n=2 each): (i) sodium ascorbate treatment at 1 g/kg for 7 hours; (ii) sodium ascorbate treatment at 2 g/kg for 7 hours; (iii) sodium ascorbate treatment at 3 g/kg for 7 hours; or (iv) fluid-matched vehicle. Norepinephrine doses were titrated to achieve a target blood pressure (75-80 mm Hg). All sodium ascorbate doses were given as a bolus followed by a continuous infusion. The bolus doses for the 1, 2 and 3 mg/kg treatments were, respectively, 0.133, 0.267 and 0.4 g/kg (5.3, 10.7 and 16 g for a 40 kg sheep. Sodium ascorbate was diluted in 1:1 ratio with 5% glucose prior to intravenous administration. At 31 hours of sepsis, animals were euthanised for tissue collection to facilitate molecular and histological investigations.

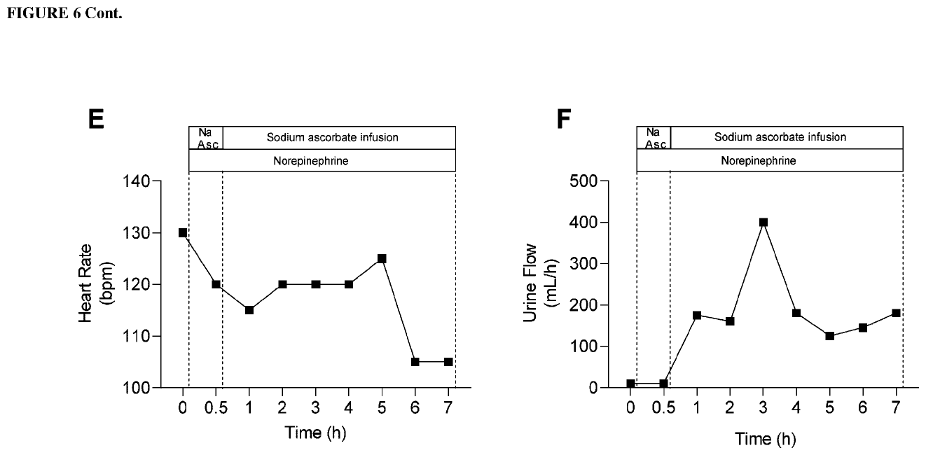

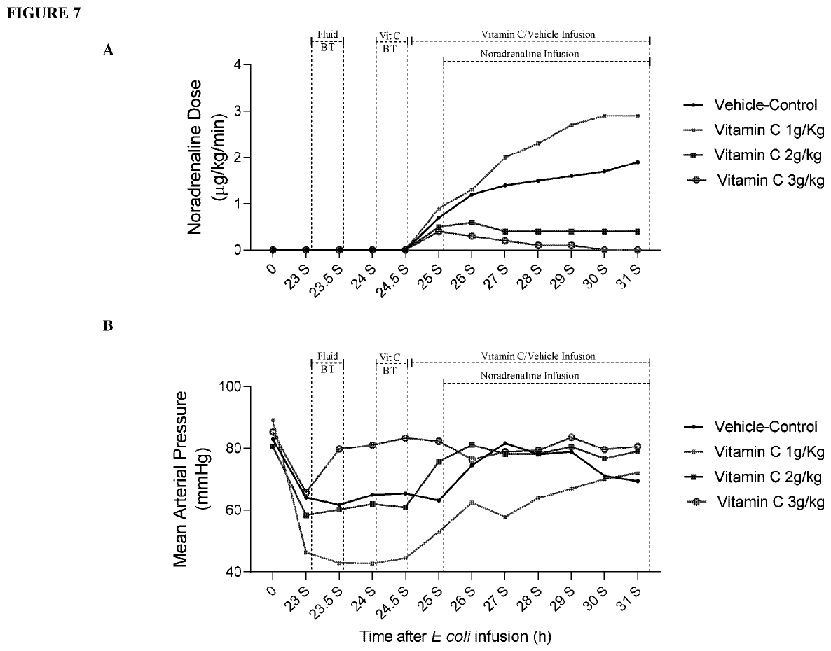

Blood pressure changes and norepinephrine dose requirements to achieve target blood pressure of 75-80 mm Hg for each of the treatment and vehicle groups are shown in FIG. 7. Sepsis is characterised by life-threatening falls in blood pressure, which necessitates clinical intervention with fluid bolus therapy followed by vasopressors to restore blood pressure and maintain hemodynamic stability in patients. The reduction in norepinephrine dose requirements with increased sodium ascorbate doses indicates that the animals are regaining vascular sensitivity to blood pressure drugs. Total withdrawal of vasopressor requirements for attaining target blood pressure was achieved with 3 g/kg sodium ascorbate.

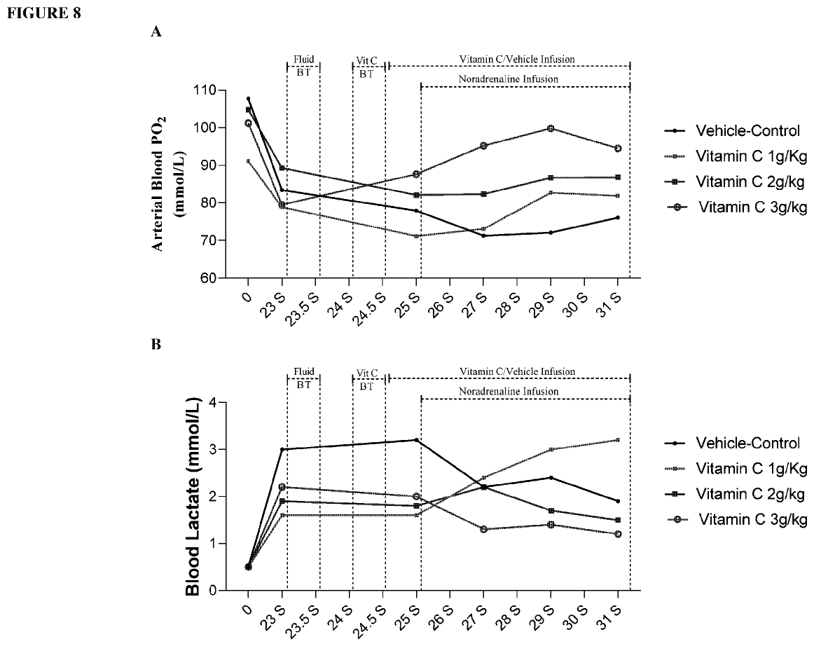

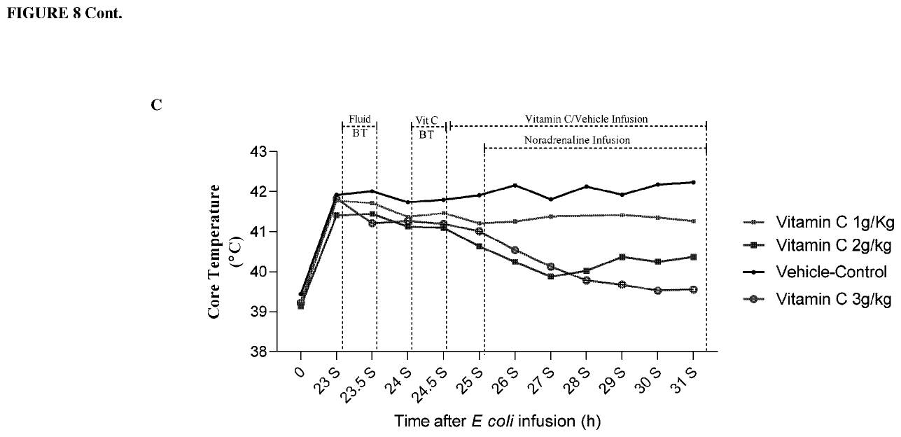

FIG. 8 shows clinical signs (arterial partial pressure of oxygen (PO₂), blood lactate levels and core temperature) for each of the treatment and vehicle groups over the course of the sepsis and treatment. A reduction in PO₂ suggests compromised lung function due to acute respiratory distress syndrome (ARDS), which is a common phenotype of sepsis. Recovery in arterial PO₂ (strongest in the 3 g/kg sodium ascorbate treatment group) is a therefore a sign of improved lung function, and has important subsequent effects towards improve arterial oxygen delivery to vital organs, which would mitigate organ dysfunction. Hyperlactatemia is a hallmark of sepsis, due to metabolic insufficiency, which occur as a result of aerobic respiration converting to anaerobic respiration likely a result of compromised lung function coupled with reduced oxygen delivery to vital organs. Accordingly, reduction in arterial blood lactate levels (greatest in the 3 g/kg sodium ascorbate treatment group) is a sign of improved metabolic function, which is associated with improved lung function and improve oxygen delivery to vital organs. High core body temperature is a hallmark of sepsis due to a systemic infection and an overwhelming inflammatory response. Normalisation of body temperature is as a clinical sign of infection resolution with sodium ascorbate (strongest in the 3 g/kg sodium ascorbate treatment group).

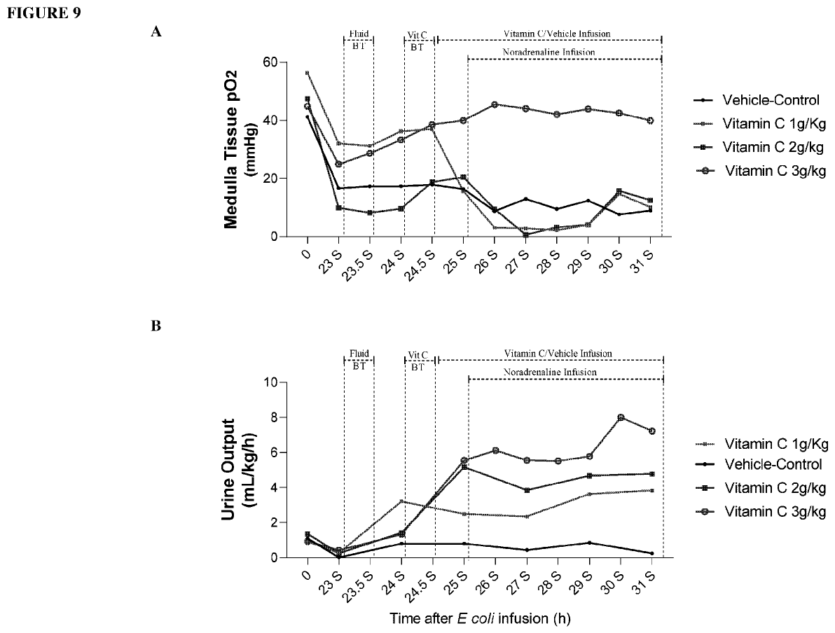

Changes in renal function (medulla tissue PO₂ and urine output over the course of sepsis and treatment for each of the sodium ascorbate and vehicle groups are shown in FIG. 9. Septic acute kidney injury (AM) is characterised by profound, selective reductions in tissue oxygen levels (hypoxia) within the inner region of the kidney (renal medulla), which the inventors suggest to be a critical driver of septic AKI. The improvement in renal medullary oxygen levels observed in the 3 g/kg sodium ascorbate treatment group is indicative of a reversal of sepsis-induced microvascular dysfunction leading to the resolution of renal medullary tissue hypoxia. Septic AKI is in part diagnosed by oliguria (reduced urinary output). An increase in urinary output (greatest in the 3 g/kg sodium ascorbate treatment group) is as an indirect clinical sign of renal functional recovery.

Based on the above dose response analysis, and in view of the observations from Examples 1 and 2, the inventors suggest that a dose of at least 3 g/kg intravenous Vitamin C is required to provide optimal benefits towards reversing the pathophysiological features of sepsis, including a reversal in cardiovascular, pulmonary, metabolic and renal dysfunction. No adverse side-effects were observed during these pre-clinical studies.

### Example 4—Human Trial of Mega-Dose Vitamin C for Septic Shock

A randomised, double-blind, placebo-controlled trial is being conducted to evaluate whether the administration of intravenous mega-dose vitamin C (60 g/day) increases urine output in patients with septic shock admitted to an intensive care unit (ICU).

Patients admitted to the Austin Hospital ICU with the primary diagnosis of septic shock defined according to the Sepsis-3 criteria are screened for eligibility. All the diagnostic criteria of septic shock (based on the SEPSIS-3 criteria) were to be fulfilled simultaneously within the last 24 hours, and vasopressors infused continuously at enrolment. Definition of sepsis is that of suspected or documented infection and an acute increase of ≥2 sequential organ failure assessment (SOFA) points consequent to the infection (a proxy of organ dysfunction). Definition of septic shock are sepsis and need for vasopressor therapy to keep mean arterial pressure (MAP) >65 mmHg for >2 hours and lactate >2 mmol/L, despite adequate fluid resuscitation.

Patients are excluded from the study if one of the following criteria presents:
Age<18 years
Pregnancy
Do not resuscitate/Do not intubate (DNR/DNI) orders
Death is deemed to be imminent or inevitable during this admission, and either the attending physician, patient or substitute decision-maker is not committed to active treatment
Patient with known HIV infection
Patient with known glucose-6 phosphate dehydrogenase (G-6PD) deficiency
Patient transferred from another ICU or hospital with a diagnosis of a septic shock for >24 hours
Patient with a diagnosis of a septic shock for >24 hours
Patient with known or suspected: history of oxalate nephropathy or hyperoxaluria; short bowel syndrome or severe fat-malabsorption; malaria; or scurvy
Patient previously enrolled in this study
Patient with chronic haemodialysis or peritoneal dialysis.
Patient requires renal replacement therapy within next 24 hours.
Patient's baseline blood sodium level is >160 mEq/L

All eligible patients with septic shock are randomised to receive either:
(i) Mega-dose Vitamin C intravenous infusion (sodium ascorbate 30 grams [100 ml] diluted with 150 ml of 5% dextrose infused intravenously over 1 hour followed by sodium ascorbate 30 grams [100 ml] diluted with 150 ml of 5% dextrose infused intravenously over 5 hours); or
(ii) Placebo intravenous infusion administered once (250 ml of 5% dextrose infused intravenously over 1 hour followed by 250 ml of 5% dextrose infused intravenously over 5 hours).

The study infusions are identical in appearance and are supplied in identical 250m1 5% dextrose bags prepared by ICU staff.

Vitamin C levels and arterial blood gas samples are measured at five time points (before loading infusion; after loading dose; 3 hours after the beginning of maintenance infusion; at the end of maintenance infusion; and, 24 hours following the beginning of the study infusion). A single urinary assessment is made of oxalate crystals obtained from testing a 24-hour collection of urine obtained at 24 hours following the beginning of the study infusion. Arterial blood gas samples are also measured to monitor for changes in serum sodium (Na+) and potassium (K+) levels at four time points (before loading infusion [baseline]; after loading dose; 3 hours after the beginning of maintenance infusion; and, at the end of maintenance infusion). The study infusion is stopped if the serum Na+ levels increases >10 mEq/L from the baseline value or absolute serum Na+ levels>160 mEq/L. Information is also collected on fluid balance, intravenous fluid therapy, use of vasopressor drugs and dosage, use of mechanical ventilation and renal replacement therapy and all biochemical and hematological and blood gas analysis variables as well as patient demographics, diagnosis, vital signs and illness severity score, ICU and hospital outcomes (e.g. admission and discharge dates as well as ICU and hospital survival status).

The primary efficacy outcome for this study is the cumulative urine output for 24 consecutive hours following the beginning of the study infusion.

Data analysis is performed on an intention-to-treat basis. Summary statistics are used to describe the clinical data and presented as mean±standard deviation, median with interquartile range (IQR) or percentages as appropriate. Chi-squared analysis with Fisher's exact test (as appropriate), and Student's t-test (Mann Whitney U test for non-normal distributions) are used to compare data between the active treatment group and the control group with statistical significance declared for probability values of less than 0.05. Analysis of the outcome of excluded patients due to other trials etc. is in accordance with CONSORT guidelines.

In the sheep study described in Example 1, urine volume increased rapidly after the intravenous mega-dose Vitamin C infusion. In the present trial, there is no safety concern signal. There is no evidence of increased oxalate excretion in the urine or increases in serum sodium levels. With vitamin C infusion, urinary output increases, requirement for vasopressor support decreases and body temperature decreases.

### REFERENCES

Calzavacca P, Evans R G, Bailey M, Bellomo R, May C N. Cortical and medullary tissue perfusion and oxygenation in experimental septic acute kidney injury. Crit Care Med 43, e431-439 (2015).
Fujii T, Luethi N, Young P J, Frei D R, Eastwood G M, French C J, et al.. Effect of Vitamin C, Hydrocortisone, and Thiamine vs Hydrocortisone Alone on Time Alive and Free of Vasopressor Support Among Patients With Septic Shock: The VITAMINS Randomized Clinical Trial. JAMA 323, 423-431 (2020).

Lankadeva Y R, Kosaka J, Evans R G, Bailey M, Bellomo R, May C N. Intra-renal and urinary oxygenation during norepinephrine resuscitation in ovine septic acute kidney injury. Kidney Int 90, 100-108 (2016).

Lankadeva Y R, Kosaka J, Evans R G, Bellomo R, May C N. Urinary oxygenation as a surrogate marker of medullary oxygenation during angiotensin II therapy in septic acute kidney injury. Crit Care Med 46, e41-48 (2018a).

Lankadeva Y R, Kosaka J, Evans R G, May C N. An ovine model for studying the pathophysiology of septic acute kidney injury. In: Traumatic and Ischemic Injury: Methods and Protocols (eds Tharakan B). Springer New York (2018b).

Marik P E, Khangoora V, Rivera R, Hooper M H, Catravas J. Hydrocortisone, Vitamin C, and Thiamine for the Treatment of Severe Sepsis and Septic Shock: A Retrospective Before—After Study. Chest 151, 1229-1238 (2017).

Osawa E A, Cutili S L, Bitker L, Canet E, Cioccari L, Iguchi N, Lankadeva Y R, Eastwood G M, Evans R G, May C N, Bellomo R, Effects of furosemide on urinary oxygenation in patients with septic shock, Blood Purification 48, 336-345 (2019).

Singer M, et al. The third international consensus definitions for sepsis and septic shock (sepsis-3). JAMA 315, 801-810 (2016).

1. A method for treating or preventing a condition, and/or symptom or clinical manifestation, associated with systemic infection in a subject, comprising intravenously administering to the subject ascorbic acid or a pharmaceutically acceptable salt, ester or isomer thereof in an amount of between about 700 mg/kg body weight and about 4,000 mg/kg body weight per day.

2-29. (canceled)
